(19) United States
(12) Patent Application Publication
(10) Pub. No.: US 2022/0202820 A1
(43) Pub. Date: Jun. 30, 2022

(54) USE OF JAK INHIBITORS FOR THE TREATMENT OF PAINFUL CONDITIONS INVOLVING NAV1.7 CHANNELS

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); FONDATION IMAGINE, Paris (FR); UNIVERSITÉ DE PARIS, Paris (FR); ASSISTANCE PUBLIQUE-HÔPITAUX DE PARIS (APHP), Paris (FR)

(72) Inventors: Christine BODEMER, Paris (FR); Patrick DELMAS, Marseille cedex 3 (FR); Céline GRECO, Paris (FR)

(21) Appl. No.: 17/604,109

(22) PCT Filed: Apr. 15, 2020
(86) PCT No.: PCT/EP2020/060543

§ 371 Date: Oct. 15, 2021

(30) Foreign Application Priority Data
Apr. 16, 2019 (EP) 19305488.9

(51) Int. Cl. A61K 31/519; A61K 31/407; A61K 31/506; A61K 31/5377; A61K 31/553; A61P 29/02

(52) U.S. Cl. CPC A61K 31/519; A61K 31/407; A61P 29/02; A61K 31/5377; A61K 31/553; A61K 31/506

(57) ABSTRACT

An increasing body of evidence suggests that Nav1.7 encoded by SCN9A gene may play a key role in various pain states, including acute, inflammatory and/or neuropathic pain. The inventors now report an efficient treatment for severe cases of primary erythromelagia linked to a specific SCN9A mutation. In particular the inventors demonstrated that the inhibition of JAK2 produces a rightward shift in the voltage dependent activation of mutant Nav1.7 channels, thereby normalizing the function of mutant Nav1.7 channels. On this basis, the inventors treated a patient suffering from PE with very severe refractory pain with a JAK2 inhibitor (ruxolitinib) and showed the therapy leads to considerable reduction of pain. Accordingly, the present invention relates to the use of JAK inhibitors for the treatment of painful conditions involving Nav1.7 channels.

### FIELD OF THE INVENTION

The present invention relates to the use of JAK inhibitors for the treatment of painful conditions involving Nav1.7 channels.

### BACKGROUND OF THE INVENTION

In the peripheral sensory nervous system the neuronal expression of voltage-gated sodium channels (Nays) is very important for the transmission of nociceptive information since they give rise to the upstroke of the action potential. Nays are composed of nine different isoforms with distinct biophysical properties.

Human SCN9A gene contains 26 exons and encodes for the α-subunit of voltage-gated sodium channel, Nav1.7 (OMIM 603415). An increasing body of evidence suggests that Nav1.7 may play a key role in various pain states, including acute, inflammatory and/or neuropathic pain.

In particular, gain of function mutations of Nav1.7 (e.g. the missense mutation (I848T)) both familial and sporadic, have been linked to primary erythermalgia (PE), a disease characterized by episodes of erythema, swelling, a painful deep-aching of the soft tissue and tenderness, along with a painful burning sensation primarily in the extremities. The incidence rate of PE ranges from 0.36 to 1.1 per 100,000 persons. The first manifestations occur usually during childhood and are frequently misdiagnosed. PE is very painful and patients report severe burning sensation with durations ranging from several minutes to hours and even days. Pain symptoms are worse in summer and at night and are usually provoked and exacerbated by heat, ambulation, physical exercise, sitting, leg dependence, coverage of extremities and stress. The severity of an intolerable and chronic pain with a regular impressive aggravation despite numerous therapeutic attempts, often lead to a progressive complete loss of the functional autonomy, sleep and appetite and disability without any social life. Almost all patients go to extreme measures to improve their symptoms, with such commonly described activities as going barefoot, keeping the house as cool as possible, using fans, cooling affected areas with cold water and ice, resting, and elevating the legs. Some reported soaking their feet in cold water for prolonged periods. Some patients described doing this for more than 20 hours a day (even while sleeping). This practice often resulted in immersion injury leading to cutaneous maceration, ulceration, and infection.

Further evidence of the role of Nav1.7 in pain is found in the phenotype of loss of function mutations of the SCN9A gene. Cox and colleagues (Nature, 444(7121):894-8 (2006)) were the first to report an association between loss-of-function mutations of SNC9A and congenital indifference to pain (CIP), a rare autosomal recessive disorder characterized by a complete indifference or insensitivity to painful stimuli.

Nav1.7 inhibitors are therefore potentially useful in the treatment of a wide range of pains. Accordingly, several drug candidates for inhibiting Nav1.7 have thus been developed (Katie Kingwell Nav1.7 withholds its pain potential, Nature Reviews Drug Discovery 2019-04-08, DOI: 10.1038/d41573-019-00065-0).

Janus kinase inhibitors are a type of drugs that functions by inhibiting the activity of one or more of the Janus kinase family of enzymes (JAK1, JAK2, JAK3, TYK2), thereby interfering with the JAK-STAT signaling pathway. These inhibitors have therapeutic application in the treatment of cancer and inflammatory diseases. There is also interest in their use for various skin conditions (Damsky, William, and Brett A. King. “JAK inhibitors in dermatology: the promise of a new drug class.” Journal of the American Academy of Dermatology 76.4 (2017): 736-744). However, the role of JAK inhibitors as Nav1.7 inhibitors has never been suggested in the prior art.

### SUMMARY OF THE INVENTION

As defined by the claims, the present invention relates to the use of JAK inhibitors for the treatment of painful conditions involving Nav1.7 channels.

### DETAILED DESCRIPTION OF THE INVENTION

The inventors now report an efficient treatment for severe cases of primary erythromelagia linked to a specific SCN9A mutation, encoding the voltage-gated sodium channel subtype Nav1.7. In particular the inventors demonstrated that the inhibition of JAK2 produces a rightward shift in the voltage dependent activation of mutant Nav1.7 channels, thereby normalizing the function of mutant Nav1.7 channels. On this basis, the inventors treated a patient suffering from PE with very severe refractory pain with a JAK2 inhibitor (ruxolitinib) and showed the therapy leads to considerable reduction of pain.

Accordingly, the present invention relates to a method of treating a painful condition involving Nav1.7 channels in a patient in need thereof comprising administering a therapeutically effective amount of a JAK inhibitor.

As used herein, the term “Nav1.7” has its general meaning in the art and refers to the sodium channel protein type 9 subunit alpha encoded by the SCN9A gene. A representative amino acid sequence for Nav1.7 is as set forth in SEQ ID NO:1. NaV1.7 is highly expressed in dorsal root ganglion (DRG) neurons and expression is further increased in DRG neurons. Evidence from studies of humans implicates NaV1.7 in pain perception. Congenital insensitivity to pain is present in persons with nonsense “loss-of-function” mutations in the gene encoding NaV1.7, (See, Cox et al (2006) Nature 444: 894-898) and chronic pain and hyperalgesia is present in persons with “gain-of-function” missense mutations in NaV1.7, such as those causing erythromelalgia, Cummings et al, (2007) Pain 131:243-257. These findings suggest that a mechanistic approach to treatment of neuropathic pain using drugs that alter the function of specific isoforms of Na+ channels (e.g., NaV1.7) is a rational therapeutic plan.

SEQ ID NO: 1
>sp|Q15858|SCN9A HUMAN Sodium channel protein
type 9 subunit alpha OS = Homo sapiens
OX = 9606 GN = SCN9A PE = 1 SV = 3
MAMLPPPGPQSFVHFIKQSLALIEQRIAERKSKEPKEEKKDDDEEAPKP
SSDLEAGKQLPFIYGDIPPGMVSEPLEDLDPYYADKKTFIVLNKGKTIF
RFNATPALYMLSPFSPLRRISIKILVHSLFSMLIMCTILINCIFMTMNN
PPDWIKNVEYTFIGIYTFESLVKILARGFCVGEFTFLRDPWNWLDFVVI
VFAYLTEFVNLGNVSALRTFRVLRALKTISVIPGLKTIVGALIQSVKKL
SDVMILTVFCLSVFALIGLQLFMGNLKHKCFRNSLENNETLESIMNTLE
SEEDFRKYFYYLEGSKDALLCGFSTDSGQCPEGYTCVKIGRNPDYGYIS
FDIFSWAFLALFRLMIQDYWENLYQQTLRAAGKTYMIFFVVVIFLGSFY
LINLILAVVAMAYEEQNQANIEEAKQKELEFQQMLDRLKKEQEEAEAIA
AAAAEYTSIRRSRIMGLSESSSETSKLSSKSAKERRNRRKKKNQKKLSS
GEEKGDAEKLSKSESEDSIRRKSFHLGVEGHRRAHEKRLSTPNQSPLSI
RGSLFSARRSSRTSLFSFKGRGRDIGSETEFADDEHSIFGDNESRRGSL
FVPHRPQERRSSNISQASRSPPMLPVNGKMHSAVDCNGVVSLVDGRSAL
MLPNGQLLPEVIIDKATSDDSGTINQIHKKRRCSSYLLSEDMLNDPNLR
QRAMSRASILTNIVEELEESRQKCPPWWYRFAHKFLIWNCSPYWIKFKK
CIYFIVMDPFVDLAITICIVLNTLFMAMEHHPMTEEFKNVLAIGNLVFI
GIFAAEMVLKLIAMDPYEYFQVGWNIFDSLIVILSLVELFLADVEGLSV
LRSERLLRVFKLAKSWPTLNMLIKIIGNSVGALGNLILVLAIIVFIFAV
VGMQLFGKSYKECVCKINDDCTLPRWHMNDFFHSFLIVERVLCGEWIET
MWDCMEVAGQAMCLIVYMMVMVIGNLVVLNLFLALLLSSFSSDNLTAIE
EDPDANNLQIAVTRIKKGINYVKQTLREFILKAFSKKPKISREIRQAED
LNIKKENYISNHTLAEMSKGHNFLKEKDKISGFGSSVDKHLMEDSDGQS
FIHNPSLIVIVPIAPGESDLENMNAEELSSDSDSEYSKVRLNRSSSSEC
STVDNPLPGEGEEAEAEPMNSDEPEACFIDGCVWRFSCCQVNIESGKGK
IWWNIRKTCYKIVEHSWFESFIVLMILLSSGALAFEDIYIERKKTIKII
LEYADKIFTYIFILEMLLKWIAYGYKTYFTNAWCWLDFLIVDVSLVTLV
ANTLGYSDLGPIKSLRILRALRPLRALSRFEGMRVVVNALIGAIPSIMN
VLLVCLIFWLIFSIMGVNLFAGKEYECINTIDGSRFPASQVPNRSECFA
LMNVSQNVRWKNLKVNFDNVGLGYLSLLQVATFKGWIIIMYAAVDSVNV
DKQPKYEYSLYMYIYFVVFIIEGSFFILNLFIGVIIDNENQQKKKLGGQ
DIFMTEEQKKYYNAMKKLGSKKPQKPIPRPGNKIQGCIFDLVINQAFDI
SIMVLICLNMVIMMVEKEGQSQHMTEVLYWINVVFIILFTGECVLKLIS
LRHYYFTVGWNIFDFVVVIISIVGMFLADLIETYFVSPILFRVIRLARI
GRILRLVKGAKGIRILLFALMMSLPALFNIGLLLFLVMFIYAIFGMSNF
AYVKKEDGINDMFNFETFGNSMICLFQITTSAGWDGLLAPILNSKPPDC
DPKKVHPGSSVEGDCGNPSVGIFYFVSYIIISFLVVVNMYIAVILENFS
VATEESTEPLSEDDFEMFYEVWEKFDPDATQFIEFSKLSDFAAALDPPL
LIAKPNKVQLIAMDLPMVSGDRIHCLDILFAFTKRVLGESGEMDSLRSQ
MEERFMSANPSKVSYEPITTILKRKQEDVSATVIQRAYRRYRLRQNVKN
ISSIYIKDGDRDDDLLNKKDMAFDNVNENSSPEKTDATSSTTSPPSYDS
VTKPDKEKYEQDRTEKEDKGKDSKESKK

As used herein, the term “painful condition involving Nav1.7 channels” thus refers to any pain or sensitivity that is caused by activation of Nav1.7 channels. Preferably, the term is understood as an abnormal sensitivity, i.e. typically as a hypersensitivity which is mediated by nociceptors. The term includes any pain selected from a nociceptor-mediated pain (also called therein a nociceptive pain), a neuropathic pain, an inflammatory pain, a pathological pain, an acute pain, a subacute pain, a chronic pain, mechanical pain, chemical pain, a somatic pain, a visceral pain, deep somatic pain, superficial somatic pain, somatoform pain, allodynia, hyperalgesia, or a pain associated with a nerve injury.

As used herein, the term “nociceptive” pain” or “nociceptor-mediated pain” occurs in response to the activation of a specific subset of peripheral sensory neurons, (nociceptors) by intense or noxious stimuli. Nociceptive pain according to the invention includes mechanical pain (e.g. crushing, tearing, etc.) and chemical pain (e.g. iodine in a cut, chili powder in the eyes). Examples of nociceptive pain include but are not limited to traumatic or surgical pain, labor pain, sprains, bone fractures, burns, bumps, bruises, injections, dental procedures, skin biopsies, and obstructions. Nociceptive pain includes visceral pain, deep somatic pain and superficial somatic pain. Visceral pain is diffuse, difficult to locate and often referred to a distant, usually superficial, structure. It may be accompanied by nausea and vomiting and may be described as sickening, deep, squeezing, and dull. Deep somatic pain is initiated by stimulation of nociceptors in ligaments, tendons, bones, blood vessels, fasciae and muscles, and is dull, aching, poorly localized pain. Examples of deep somatic pain include sprains and broken bones. Superficial pain is initiated by activation of nociceptors in the skin or other superficial tissue, and is sharp, well-defined and clearly located. Examples of injuries that produce superficial somatic pain include minor wounds and minor (first degree) burns. Inflammatory pain is pain that occurs in the presence of tissue damage or inflammation including postoperative, post-traumatic pain, arthritic (rheumatoid or osteoarthritis) pain and pain associated with damage to joints, muscle, and tendons as in axial low back pain. Inflammation is responsible for the sensitization of peripheral sensory neurons, leading to spontaneous pain and invalidating pain hypersensitivity. Acute or chronic pathological tissue inflammation strongly impacts on pain perception by sensitizing peripheral sensory neurons, giving rise to local and incapacitating pain hypersensitivity. Inflammatory mediators are known to enhance nociceptive primary afferent fibers excitability, in part by modifying expression and/or function of ion channels present in nerve endings.

Neuropathic pain is a common type of chronic, non-malignant pain, which is the result of an injury or malfunction in the peripheral or central nervous system. Neuropathic pain may have different etiologies, and may occur, for example, due to trauma, surgery, herniation of an intervertebral disk, spinal cord injury, diabetes, infection with herpes zoster (shingles), HIV/AIDS, late-stage cancer, amputation (including mastectomy), carpal tunnel syndrome, chronic alcohol use, exposure to radiation, and as an unintended side-effect of neurotoxic treatment agents, such as certain anti-HIV and chemotherapeutic drugs. It is often characterized by chronic allodynia (defined as pain resulting from a stimulus that does not ordinarily elicit a painful response, such as light touch) and hyperalgesia (defined as an increased sensitivity to a normally painful stimulus), and may persist for months or years beyond the apparent healing of any damaged tissues.

Pain may also occur in patients with cancer, which may be due to multiple causes; inflammation, compression, invasion, metastatic spread into bone or other tissues. Pain also includes migraine and a headache associated with the activation of sensory fibers innervating the meninges of the brain.

In some embodiments, the painful condition is caused by a gain of function mutation in SCN9A. Typically, said mutation is selected from the group consisting of Q10R, F216S, S241T, N395K, E406K, I859T, L869F, L869H, F1460V, A1643E and A1643T in SEQ ID NO: 1.

Thus in some embodiments, the JAK inhibitor of the present invention is particularly suitable for the treatment of primary erythermalgia. As used herein, the term “primary erythermalgia” has its general meaning in the art and refers to an autosomal dominant disorder characterized by childhood or adolescent onset of episodic symmetrical red congestion, vasodilatation, and burning pain of the feet and lower legs provoked by exercise, long standing, and exposure to warmth. The severity of the disorder may progress with age, and symptoms may extend over a larger body area, such as over the ankles and lower legs, and become constant.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein the term “JAK” has its general meaning in the art and refers to the family of Janus kinases (JAKs) which are cytoplasmic tyrosine kinases that transduce cytokine signaling from membrane receptors to STAT transcription factors. Four JAK family members are described, JAK1, JAK2, JAK3 and TYK2 and the term JAK may refer to all the JAK family members collectively or one or more of the JAK family members as the context indicates.

As used herein the term “JAK inhibitor” is intended to mean compounds inhibit the activity or expression of at least JAK2. JAK inhibitors down-regulate the quantity or activity of JAK molecules. One activity of JAK2 is to phosphorylate a STAT protein. Therefore an example of an effect of a JAK inhibitor is to decrease the phosphorylation of one or more STAT proteins. The inhibitor may inhibit the phosphorylated form of JAK2 or the non-phosphorylated form of JAK2. In some embodiments, the JAK inhibitor is a selective JAK2 inhibitor. By “selective” is meant that the compound binds to or inhibits JAK2 with greater affinity or potency, respectively, compared to at least one other JAK (e.g., JAK1, JAK3 and/or TYK2). Selectivity can be at least about 5-fold, at least about 10-fold, at least about 20-fold, at least about 50-fold, at least about 100-fold, at least about 200-fold, at least about 500-fold or at least about 1000-fold. Selectivity can be measured by methods routine in the art. In some embodiments, selectivity can be tested at the Km of each enzyme. In some embodiments, selectivity of compounds for JAK2 can be determined by the cellular ATP concentration.

JAK inhibitors are well known in the art. For example, JAK inhibitors include phenylaminopyrimidine compounds (WO2009/029998), substituted tricyclic heteroaryl compounds (WO2008/079965), cyclopentyl-propanenitrile compounds (WO2008/157208 and WO2008/157207), indazole derivative compounds (WO2008/114812), substituted ammo-thiophene carboxylic acid amide compounds (WO2008/156726), naphthyridine derivative compounds (WO2008/112217), quinoxaline derivative compounds (WO2008/148867), pyrrolopyrimidine derivative compounds (WO2008/119792), purinone and imidazopyridinone derivative compounds (WO2008/060301), 2,4-pyrimidinediamine derivative compounds (WO2008/118823), deazapurine compounds (WO2007/117494) and tricyclic heteroaryl compounds (WO2008/079521). Examples of JAK inhibitors include compounds disclosed in the following publications: US2004/176601, US2004/038992, US2007/135466, US2004/102455, WO2009/054941, US2007/134259, US2004/265963, US2008/194603, US2007/207995, US2008/260754, US2006/063756, US2008/261973, US2007/142402, US2005/159385, US2006/293361, US2004/205835, WO2008/148867, US2008/207613, US2008/279867, US2004/09799, US2002/055514, US2003/236244, US2004/097504, US2004/147507, US2004/176271, US2006/217379, US2008/092199, US2007/043063, US2008/021013, US2004/152625, WO2008/079521, US2009/186815, US2007/203142, WO2008/144011, US2006/270694 and US2001/044442. JAK inhibitors further include compounds disclosed in the following publications: WO2003/011285, WO2007/145957, WO2008/156726, WO2009/035575, WO2009/054941, and WO2009/075830. JAK inhibitors further include compounds disclosed in the following patent applications: U.S. Ser. Nos. 61/137,475 and 61/134,338.

Specific JAK inhibitors include AG490, AUB-6-96, AZ960, AZD1480, baricitinib (LY3009104, INCB28050), BMS-911543, CEP-701, CMP6, CP352,664, CP690,550, CYT-387, INCB20, Jak2-IA, lestaurtinib (CEP-701), LS104, LY2784544, NS018, pacritinib (SB1518), Pyridone 6, ruxolitinib (INCB018424), SB1518, TG101209, TG101348 (SAR302503), TG101348, tofacitinib (CP-690,550), WHI-PI 54, WP1066, XL019, and XLOI 9. Ruxolitinib (Jakafi™, INCB018424; (3R)-3-cyclopentyl-3-[4-(7H-pyrrolo[2,3-d]pyrimidin-4-yl)pyrazol-1-yl]propanenitrile) is a potent, orally available, selective inhibitor of both JAK1 and JAK2 of the JAK-STAT signaling pathway. CYT387 is an inhibitor of Janus kinases JAK1 and JAK2, acting as an ATP competitor with IC50 values of 11 and 18 nM, respectively. TG101348 (SAR302503) is an orally available inhibitor of Janus kinase 2 (JAK-2). TG101348 acts as a competitive inhibitor of protein kinase JAK-2 with IC50=6 nM; related kinases FLT3 and RET are also sensitive, with IC50=25 nM and IC50=17 nM, respectively. AZD1480 is an orally bioavailable inhibitor of Janus-associated kinase 2 (JAK2) with potential antineoplastic activity. JAK2 inhibitor AZD 1480 inhibits JAK2 activation, leading to the inhibition of the JAK/STAT (signal transducer and activator of transcription) signaling including activation of STAT3. Lestaurtinib (CEP-701) is a tyrosine kinase inhibitor structurally related to staurosporine. Pacritinib (SB 1815) is an orally bioavailable inhibitor of JAK2 and the JAK2 mutant JAK2V617F. Pacritinib competes with JAK2 for ATP binding, which may result in inhibition of JAK2 activation, inhibition of the JAK-STAT signaling pathway, and therefore caspase-dependent apoptosis. Baricitinib (LY3009104, INCB28050) is an orally bioavailable inhibitor of JAK1 and JAK2 with IC50=5.9 nm and IC50=5.7, nm respectively. Baricitinib preferentially inhibits JAK1 and JAK2, with 10-fold selectivity over Tyk2 and 100-fold over JAK3. XL019 is an orally bioavailable inhibitor of Janus-associated kinase 2 (JAK2). XL019 inhibits the activation of JAK2 as well as the mutated form JAK2V617F. NS018 is a potent JAK2 inhibitor with some inhibition of Src-family kinases. NS018 has been shown to be highly active against JAK2 with a 50% inhibition (IC50) of <1 nM, and had 30-50-fold greater selectivity for JAK2 over other JAK-family kinases.

Selective JAK2 inhibitors are well known in the art and are typically described in the following publications:
1: Lin T E, HuangFu W C, Chao M W, Sung T Y, Chang C D, Chen Y Y, Hsieh J H, Tu H J, Huang H L, Pan S L, Hsu K C. A Novel Selective JAK2 Inhibitor Identified Using Pharmacological Interactions. Front Pharmacol. 2018 Dec. 4; 9:1379. doi: 10.3389/fphar.2018.01379. eCollection 2018. PubMed PMID: 30564118; PubMed Central PMCID: PMC6288363.
2: Wan H, Schroeder G M, Hart A C, Inghrim J, Grebinski J, Tokarski J S, Lorenzi M V, You D, Mcdevitt T, Penhallow B, Vuppugalla R, Zhang Y, Gu X, Iyer R, Lombardo L J, Trainor G L, Ruepp S, Lippy J, Blat Y, Sack J S, Khan J A, Stefanski K, Sleczka B, Mathur A, Sun J H, Wong M K, Wu D R, Li P, Gupta A, Arunachalam P N, Pragalathan B, Narayanan S, K C N, Kuppusamy P, Purandare A V. Discovery of a Highly Selective JAK2 Inhibitor, BMS-911543, for the Treatment of Myeloproliferative Neoplasms. ACS Med Chem Lett. 2015 Jul. 12; 6(8):850-5. doi: 10.1021/acsmedchemlett.5b00226. eCollection 2015 Aug. 13. PubMed PMID: 26288683; PubMed Central PMCID: PMC4538448.
3: Kuroda J, Kodama A, Chinen Y, Shimura Y, Mizutani S, Nagoshi H, Kobayashi T, Matsumoto Y, Nakaya Y, Tamura A, Kobayashi Y, Naito H, Taniwaki M. NS-018, a selective JAK2 inhibitor, preferentially inhibits CFU-GM colony formation by bone marrow mononuclear cells from high-risk myelodysplastic syndrome patients. Leuk Res. 2014 May; 38(5):619-24. doi: 10.1016/j.leukres.2014.03.001. Epub 2014 Mar. 11. PubMed PMID: 24679585.
4: Verstovsek S, Tam C S, Wadleigh M, Sokol L, Smith C C, Bui L A, Song C, Clary D O, Olszynski P, Cortes J, Kantarjian H, Shah N P. Phase I evaluation of XL019, an oral, potent, and selective JAK2 inhibitor. Leuk Res. 2014 March; 38(3):316-22. doi: 10.1016/j.leukres.2013.12.006. Epub 2013 Dec. 11. PubMed PMID: 24374145; PubMed Central PMCID: PMC4414320.
5: Dugan B J, Gingrich D E, Mesaros E F, Milkiewicz K L, Curry M A, Zulli A L, Dobrzanski P, Serdikoff C, Jan M, Angeles T S, Albom M S, Mason J L, Aimone L D, Meyer S L, Huang Z, Wells-Knecht K J, Ator M A, Ruggeri B A, Dorsey B D. A selective, orally bioavailable 1,2,4-triazolo[1,5-a]pyridine-based inhibitor of Janus kinase 2 for use in anticancer therapy: discovery of CEP-33779. J Med Chem. 2012 Jun. 14; 55(11):5243-54. doi: 10.1021/jm300248q. Epub 2012 May 18. PubMed PMID: 22594690.
6: Seavey M M, Lu L D, Stump K L, Wallace N H, Hockeimer W, O'Kane T M, Ruggeri B A, Dobrzanski P. Therapeutic efficacy of CEP-33779, a novel selective JAK2 inhibitor, in a mouse model of colitis-induced colorectal cancer. Mol Cancer Ther. 2012 April; 11(4):984-93. doi: 10.1158/1535-7163.MCT-11-0951. Epub 2012 Feb. 14. PubMed PMID: 22334590.
7: Purandare A V, McDevitt T M, Wan H, You D, Penhallow B, Han X, Vuppugalla R, Zhang Y, Ruepp S U, Trainor G L, Lombardo L, Pedicord D, Gottardis M M, Ross-Macdonald P, de Silva H, Hosbach J, Emanuel S L, Blat Y, Fitzpatrick E, Taylor T L, McIntyre K W, Michaud E, Mulligan C, Lee F Y, Woolfson A, Lasho T L, Pardanani A, Tefferi A, Lorenzi M V. Characterization of BMS-911543, a functionally selective small-molecule inhibitor of JAK2. Leukemia. 2012 February; 26(2):280-8. doi: 10.1038/leu.2011.292. Epub 2011 Oct. 21. PubMed PMID:22015772.
8: Shide K, Kameda T, Markovtsov V, Shimoda H K, Tonkin E, Fang S, Liu C, Gelman M, Lang W, Romero J, McLaughlin J, Bhamidipati S, Clough J, Low C, Reitsma A, Siu S, Pine P, Park G, Torneros A, Duan M, Singh R, Payan D G, Matsunaga T, Hitoshi Y, Shimoda K. R723, a selective JAK2 inhibitor, effectively treats JAK2V617F-induced murine myeloproliferative neoplasm. Blood. 2011 Jun. 23; 117(25):6866-75. doi: 10.1182/blood-2010-01-262535. Epub 2011 Apr. 29. PubMed PMID: 21531978.
9: Pardanani A, Gotlib J R, Jamieson C, Cortes J E, Talpaz M, Stone R M, Silverman M H, Gilliland D G, Shorr J, Tefferi A. Safety and efficacy of TG101348, a selective JAK2 inhibitor, in myelofibrosis. J Clin Oncol. 2011 Mar. 1; 29(7):789-96. doi: 10.1200/JCO.2010.32.8021. Epub 2011 Jan. 10. PubMed PMID: 21220608; PubMed Central PMCID: PMC4979099.

In some embodiments, the selective JAK2 inhibitor is selected from the group consisting of Fedratinib, Gandotinib, Lestaurtinib, and Pacritinib.

In some embodiments, the JAK inhibitor is an inhibitor of expression of JAK2. An “inhibitor of expression” refers to a natural or synthetic compound that has a biological effect to inhibit the expression of a gene. In some embodiments, said inhibitor of gene expression is a siRNA, an antisense oligonucleotide or a ribozyme. For example, anti-sense oligonucleotides, including anti-sense RNA molecules and anti-sense DNA molecules, would act to directly block the translation of mRNA by binding thereto and thus preventing protein translation or increasing mRNA degradation, thus decreasing the level of the protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. Gene expression can be reduced by contacting a subject or cell with a small double stranded RNA (dsRNA), or a vector or construct causing the production of a small double stranded RNA, such that JAK2 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cell. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

By a “therapeutically effective amount” of the JAK inhibitor as above described is meant a sufficient amount of the compound. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific inhibitor employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the JAK inhibitor of the present invention is combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In some embodiments, it may be desirable to administer the JAK inhibitor of the present invention in a topical formulation. As used herein the term “topical formulation” refers to a formulation that may be applied to skin. Topical formulations can be used for both topical and transdermal administration of substances. As used herein, “topical administration” is used in its conventional sense to mean delivery of a substance, such as a therapeutically active agent, to the skin or a localized region of a subject's body. As used herein, “transdermal administration” refers to administration through the skin. Transdermal administration is often applied where systemic delivery of an active is desired, although it may also be useful for delivering an active to tissues underlying the skin with minimal systemic absorption. Typically, the topical pharmaceutically acceptable carrier is any substantially nontoxic carrier conventionally usable for topical administration of pharmaceuticals in which the JAK inhibitor of the present invention will remain stable and bioavailable when applied directly to skin surfaces. For example, carriers such as those known in the art effective for penetrating the keratin layer of the skin into the stratum comeum may be useful in delivering the JAK inhibitor of the present invention to the area of interest. Such carriers include liposomes. JAK inhibitor of the present invention can be dispersed or emulsified in a medium in a conventional manner to form a liquid preparation or mixed with a semi-solid (gel) or solid carrier to form a paste, powder, ointment, cream, lotion or the like. Suitable topical pharmaceutically acceptable carriers include water, buffered saline, petroleum jelly (vaseline), petrolatum, mineral oil, vegetable oil, animal oil, organic and inorganic waxes, such as microcrystalline, paraffin and ozocerite wax, natural polymers, such as xanthanes, gelatin, cellulose, collagen, starch, or gum arabic, synthetic polymers, alcohols, polyols, and the like. The carrier can be a water miscible carrier composition. Such water miscible, topical pharmaceutically acceptable carrier composition can include those made with one or more appropriate ingredients outset of therapy. The topical acceptable carrier will be any substantially non-toxic carrier conventionally usable for topical administration in which JAK inhibitor of the present invention will remain stable and bioavailable when applied directly to the skin surface. Suitable cosmetically acceptable carriers are known to those of skill in the art and include, but are not limited to, cosmetically acceptable liquids, creams, oils, lotions, ointments, gels, or solids, such as conventional cosmetic night creams, foundation creams, suntan lotions, sunscreens, hand lotions, make-up and make-up bases, masks and the like. Any suitable carrier or vehicle effective for topical administration to a patient as known in the art may be used, such as, for example, a cream base, creams, liniments, gels, lotions, ointments, foams, solutions, suspensions, emulsions, pastes, aqueous mixtures, sprays, aerosolized mixtures, oils such as Crisco®, soft-soap, as well as any other preparation that is pharmaceutically suitable for topical administration on human and/or animal body surfaces such as skin or mucous membranes. Topical acceptable carriers may be similar or identical in nature to the above described topical pharmaceutically acceptable carriers. It may be desirable to have a delivery system that controls the release of JAK inhibitor of the present invention to the skin and adheres to or maintains itself on the skin for an extended period of time to increase the contact time of the JAK inhibitor of the present invention on the skin. Sustained or delayed release of JAK inhibitor of the present invention provides a more efficient administration resulting in less frequent and/or decreased dosage of JAK inhibitor of the present invention and better patient compliance. Examples of suitable carriers for sustained or delayed release in a moist environment include gelatin, gum arabic, xanthane polymers. Pharmaceutical carriers capable of releasing the JAK inhibitor of the present invention when exposed to any oily, fatty, waxy, or moist environment on the area being treated, include thermoplastic or flexible thermoset resin or elastomer including thermoplastic resins such as polyvinyl halides, polyvinyl esters, polyvinylidene halides and halogenated polyolefins, elastomers such as brasiliensis, polydienes, and halogenated natural and synthetic rubbers, and flexible thermoset resins such as polyurethanes, epoxy resins and the like. Controlled delivery systems are described, for example, in U.S. Pat. No. 5,427,778 which provides gel formulations and viscous solutions for delivery of the JAK inhibitor of the present invention to a skin site. Gels have the advantages of having a high water content to keep the skin moist, the ability to absorb skin exudate, easy application and easy removal by washing. Preferably, the sustained or delayed release carrier is a gel, liposome, microsponge or microsphere. The JAK inhibitor of the present invention can also be administered in combination with other pharmaceutically effective agents including, but not limited to, antibiotics, other skin healing agents, and antioxidants. In some embodiments, the topical formulation of the present invention comprises a penetration enhancer. As used herein, “penetration enhancer” refers to an agent that improves the transport of molecules such as an active agent (e.g., a drug) into or through the skin. Various conditions may occur at different sites in the body either in the skin or below creating a need to target delivery of compounds. Thus, a “penetration enhancer” may be used to assist in the delivery of an active agent directly to the skin or underlying tissue or indirectly to the site of the disease or a symptom thereof through systemic distribution. A penetration enhancer may be a pure substance or may comprise a mixture of different chemical entities.

The invention will be further illustrated by the following FIGURES and examples. However, these examples and FIGURES should not be interpreted in any way as limiting the scope of the present invention.

### FIGURES

FIG. 1. AG490 normalizes the properties of I848T mutant Nav1.7 channels. Normalized conductance-voltage curves for WT mNav1.7 (open circles) and I848T mutants pretreated (blue symbols) or not (red symbols) with 10 μM AG490 (10 min). Curves were fitted by using a first-order Boltzmann relation. V_{1/2}=−31.44±1 mV (I848T), −22.13±0.5 mV (WT), −26.32±0.6 mV (I848T+AG490). *P=0.032, I848T+AG490 vs. I848T; data shown as means±SEM (n=5−6). Inset: Nav1.7 currents were evoked by depolarizing pulses from −80 to 20 mV, while the cell was hold at −100 mV.

### EXAMPLE

A number of studies on primary erythermalgia have elucidated a close relationship between gain of function of Nav1.7 and hyperexcitability of peripheral nociceptive neurons. To characterize the functional impact of the I848T mutation, mutant Nav1.7 cDNA was expressed in Human embryonic kidney (HEK) 293 cells, and Nav1.7 current was recorded using the whole-cell patch clamp technique (See Methods).

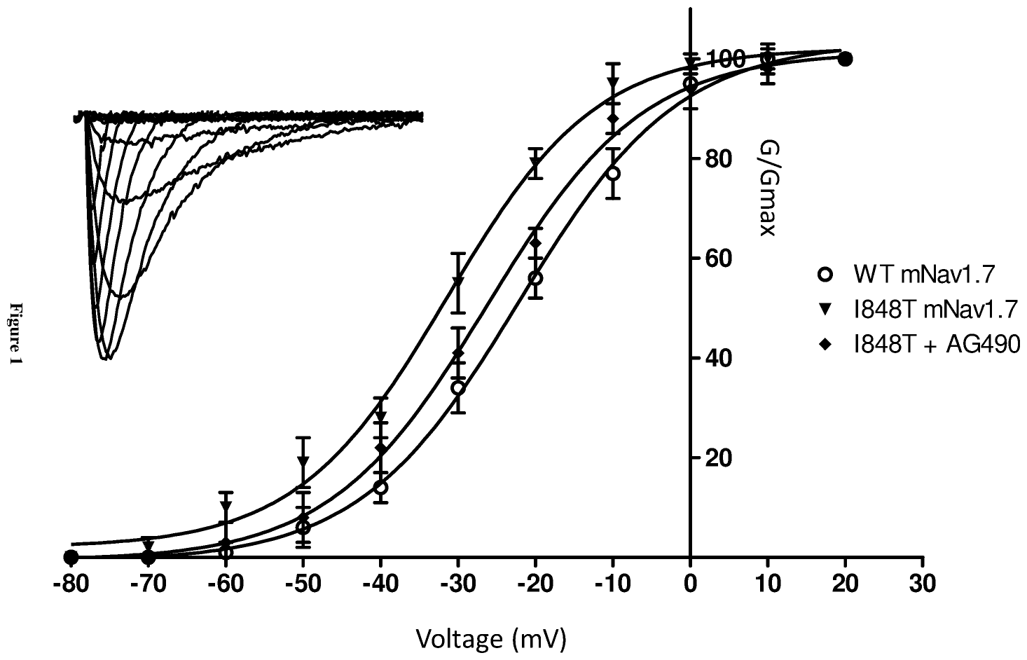

The current-voltage relationships of wild type and mutant I848T Nav1.7 channels were recorded. Cells were held at −100 mV and stepped to a range of potentials (−80 to 20 mV with 5 mV increment) for 50 ms to record the current amplitude that was induced at each voltage step (inset in FIG. 1). The current-voltage curve of I848T mutant showed that there is a hyperpolarizing shift in the voltage dependent activation for mutant channels with no apparent shift in reversal potential (not shown). To obtain activation curves, normalized conductance was fitted with Boltzmann equation, and the half maximal activation potential (V_{1/2}) of each channel was calculated (FIG. 1). I848T mutant channels display a significant (P<0.05) hyperpolarizing shift in activation V_{1/2} (I848T: −31.44±1 mV, n=5) compared with wild type channels (WT: −22.13±0.5 mV, n=5). The slope factors of activation curves are similar. We examined the voltage dependence of I848T mutant channel activation in HEK cells pre-treated with AG490 (10 μM), a specific inhibitor of the Janus tyrosine kinase 2 (JAK2), for 10 min. AG490 induced a positive shift in the activation curve of mutant channels, giving a half-activation voltage of −26.32±0.6 mV (n=6).

Collectively, these results indicate that the inhibition of JAK2 by AG490 produces a rightward shift in the voltage dependent activation of mutant Nav1.7 channels, thereby normalizing the function of mutant Nav1.7 channels.

Thanks to these fundamental data, we decided to treat a patient suffering from PE with very severe refractory pain.

Our patient, a man in his 30s suffered from intractable pain in feet due to PE (gain of function mutation c.2543T>C in the SCN9A gene) leading to major impact on his general condition (malnutrition, depressive syndrome, insomnia, social isolation, severe skin lesion due to prolonged immersion in cold water or ice). He scored 6/10 on the “Douleur Neuropathique 4” (DN4) questionnaire and 9/10 on the Visual Analog Scale for pain (VAS). All conventional treatments had been tried (opioids, anti-epileptic and anti-depressants drugs, ketamine, mexiletine, calcium channel blockers, beta-blockers, clonidine, botulinum toxin, capsaicin plaster, methadone, etc. . . . ) with no efficacy. He described intense “pain attacks” during which he experienced pulsatile pain and felt “stabs”. During these painful episodes, edemas of the hands and feet are observed with redness and local warmth.

After one month of treatment with Ruxolitinib 10 mg t.i.d, VAS pain was evaluated at 0/10 most of the time with some seizures where the intensity of the pain reaches 2/10. We observed the disappearance of edemas during seizures as well as redness and local heat. Only persists, according to the patient a small area of redness next to the external malleoli.

In conclusion, this is the first report of an efficient treatment for severe cases of primary erythromelagia linked to a specific SCN9A mutation. We have the perspective to treat other adults patients but also children considering the potential severity since childhood with a real “no-life” for the patients and severe secondary complications (delay to growth, psychological and physical alteration, denutrition . . . ) related to pain. Our objective is also to have a better understanding of the modulation of the pain with JAK inhibitors, very useful for other painful conditions involving Naᵥ1.7 channels

### REFERENCES

Throughout this application, various references describe the state of the art to which this invention pertains. The disclosures of these references are hereby incorporated by reference into the present disclosure.
Dib-Hajj S D, Rush A M, Cummins T R, Hisama F M, Novella S, Tyrrell L, et al. (2005). Gain-of-function mutation in Nav1.7 in familial erythromelalgia induces bursting of sensory neurons. Brain 128:1847-54.
Hao J, Padilla F, Dandonneau M, Lavebratt C, Lesage F, Delmas, P. (2013). Kv1.1 channels act as mechanical brake in the senses of touch and pain. Neuron 77:899-914
Waxman S. and Dib-Hajj S D, (2005). Molecular basis for an inherited pain syndrome, Trends in Molecular Medicine, 11(12), 555-562.
Friberg D, Chen T, Tarr G, van Rij A. Erythromelalgia? A clinical study of people who experience red, hot, painful feet in the community. International journal of vascular medicine. 2013; 2013:864961.
Tang Z, Chen Z, Tang B, Jiang H. Primary erythromelalgia: a review. Orphanet journal of rare diseases. Sep. 30, 2015; 10:127.
Skeik N, Rooke T W, Davis M D, et al. Severe case and literature review of primary erythromelalgia: novel SCN9A gene mutation. Vascular medicine (London, England). February 2012; 17(1):44-49.
Bennett D L, Woods C G. Painful and painless channelopathies. The Lancet. Neurology. June 2014; 13(6):587-599.
Herzog R I, Cummins T R, Waxman S G. Persistent TTX-resistant Na+ current affects resting potential and response to depolarization in simulated spinal sensory neurons. Journal of neurophysiology. September 2001; 86(3):1351-1364.
Dib-Hajj S D, Yang Y, Black J A, Waxman S G. The Na(V)1.7 sodium channel: from molecule to man. Nature reviews. Neuroscience. January 2013; 14(1):49-62.
Dib-Hajj S D, Cummins T R, Black J A, Waxman S G. From genes to pain: Na v 1.7 and human pain disorders. Trends in neurosciences. November 2007; 30(11):555-563.

1. A method of treating a painful condition involving Nav1.7 channels in a patient in need thereof comprising administering a therapeutically effective amount of a JAK inhibitor.

2. The method of claim 1 wherein the painful condition is a nociceptive pain, a neuropathic pain, an inflammatory pain, a pathological pain, an acute pain, a subacute pain, a chronic pain, mechanical pain, chemical pain, a somatic pain, a visceral pain, deep somatic pain, superficial somatic pain, somatoform pain, allodynia, hyperalgesia, or a pain associated with a nerve injury.

3. The method of claim 2 wherein the nociceptive pain includes visceral pain, deep somatic pain and superficial somatic pain.

4. The method of claim 1 wherein the painful condition is caused by a gain of function mutation in SCN9A.

5. The method of claim 7 the mutation is selected from the group consisting of Q10R, F216S, S241T, N395K, E406K, I859T, L869F, L869H, F1460V, A1643E and A1643T in SEQ ID NO: 1.

6. The method of claim 4 wherein the painful condition is primary erythermalgia.

7. The method of claim 1 wherein the JAK inhibitor is ruxolitinib.

8. The method of claim 1 wherein JAK inhibitor is a selective JAK2 inhibitor.

9. The method of claim 8 wherein the selective JAK2 inhibitor is selected from the group consisting of Fedratinib, Gandotinib, Lestaurtinib, and Pacritinib.

10. The method of claim 1 wherein the JAK inhibitor is administered to the patient in a form of a topical formulation.


SEQUENCE LISTING (1 sequence)

SEQ ID NO: 1 - protein, 1988 aa, Homo sapiens
MAMLPPPGPQSFVHFTKQSLALIEQRIAERKSKEPKEEKKDDDEEAPKPSSDLEAGKQLPFIYGDIPPGMVSEPLEDLDPYYADKKTFIVLNKGKTIFRFNATPALYMLSPFSPLRRISIKILVHSLFSMLIMCTILTNCIFMTMNNPPDWTKNVEYTFTGIYTFESLVKILARGFCVGEFTFLRDPWNWLDFVVIVFAYLTEFVNLGNVSALRTFRVLRALKTISVIPGLKTIVGALIQSVKKLSDVMILTVFCLSVFALIGLQLFMGNLKHKCFRNSLENNETLESIMNTLESEEDFRKYFYYLEGSKDALLCGFSTDSGQCPEGYTCVKIGRNPDYGYTSFDTFSWAFLALFRLMTQDYWENLYQQTLRAAGKTYMIFFVVVIFLGSFYLINLILAVVAMAYEEQNQANIEEAKQKELEFQQMLDRLKKEQEEAEAIAAAAAEYTSIRRSRIMGLSESSSETSKLSSKSAKERRNRRKKKNQKKLSSGEEKGDAEKLSKSESEDSIRRKSFHLGVEGHRRAHEKRLSTPNQSPLSIRGSLFSARRSSRTSLFSFKGRGRDIGSETEFADDEHSIFGDNESRRGSLFVPHRPQERRSSNISQASRSPPMLPVNGKMHSAVDCNGVVSLVDGRSALMLPNGQLLPEVIIDKATSDDSGTTNQIHKKRRCSSYLLSEDMLNDPNLRQRAMSRASILTNTVEELEESRQKCPPWWYRFAHKFLIWNCSPYWIKFKKCIYFIVMDPFVDLAITICIVLNTLFMAMEHHPMTEEFKNVLAIGNLVFTGIFAAEMVLKLIAMDPYEYFQVGWNIFDSLIVTLSLVELFLADVEGLSVLRSFRLLRVFKLAKSWPTLNMLIKIIGNSVGALGNLTLVLAIIVFIFAVVGMQLFGKSYKECVCKINDDCTLPRWHMNDFFHSFLIVFRVLCGEWIETMWDCMEVAGQAMCLIVYMMVMVIGNLVVLNLFLALLLSSFSSDNLTAIEEDPDANNLQIAVTRIKKGINYVKQTLREFILKAFSKKPKISREIRQAEDLNTKKENYISNHTLAEMSKGHNFLKEKDKISGFGSSVDKHLMEDSDGQSFIHNPSLTVTVPIAPGESDLENMNAEELSSDSDSEYSKVRLNRSSSSECSTVDNPLPGEGEEAEAEPMNSDEPEACFTDGCVWRFSCCQVNIESGKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMRVVVNALIGAIPSIMNVLLVCLIFWLIFSIMGVNLFAGKFYECINTTDGSRFPASQVPNRSECFALMNVSQNVRWKNLKVNFDNVGLGYLSLLQVATFKGWTIIMYAAVDSVNVDKQPKYEYSLYMYIYFVVFIIFGSFFTLNLFIGVIIDNFNQQKKKLGGQDIFMTEEQKKYYNAMKKLGSKKPQKPIPRPGNKIQGCIFDLVTNQAFDISIMVLICLNMVTMMVEKEGQSQHMTEVLYWINVVFIILFTGECVLKLISLRHYYFTVGWNIFDFVVVIISIVGMFLADLIETYFVSPTLFRVIRLARIGRILRLVKGAKGIRTLLFALMMSLPALFNIGLLLFLVMFIYAIFGMSNFAYVKKEDGINDMFNFETFGNSMICLFQITTSAGWDGLLAPILNSKPPDCDPKKVHPGSSVEGDCGNPSVGIFYFVSYIIISFLVVVNMYIAVILENFSVATEESTEPLSEDDFEMFYEVWEKFDPDATQFIEFSKLSDFAAALDPPLLIAKPNKVQLIAMDLPMVSGDRIHCLDILFAFTKRVLGESGEMDSLRSQMEERFMSANPSKVSYEPITTTLKRKQEDVSATVIQRAYRRYRLRQNVKNISSIYIKDGDRDDDLLNKKDMAFDNVNENSSPEKTDATSSTTSPPSYDSVTKPDKEKYEQDRTEKEDKGKDSKESKK